(19) United States
(12) Patent Application Publication
(10) Pub. No.: US 2011/0046734 A1
(43) Pub. Date: Feb. 24, 2011

(54) Ligament And Tendon Prosthesis

(75) Inventors: Idan Tobis, Beit Hashmonai (IL); Nir Tobis, Beit Hashmonaim (IL)

Correspondence Address: LERNER, DAVID, LITTENBERG,;KRUMHOLZ & MENTLIK, 600 SOUTH AVENUE WEST, WESTFIELD, NJ, 07090, US

(73) Assignee: TAVOR (I.T.N) LTD., Ashquelon (IL)

(21) Appl. No.: 12/922,012

(22) PCT Filed: Mar. 15, 2009
(86) PCT No.: PCT/IL09/00291

§ 371 Date: Nov. 5, 2010

Related U.S. Application Data
(60) Provisional application No. 61/064,584, filed on Mar. 13, 2008.

(51) Int. Cl. A61F 2/08

(52) U.S. Cl. 623/13.14; 623/13.11

(57) ABSTRACT

The invention provides a tendon or ligament prosthesis having an undeployed configuration and a deployed configuration. The prosthesis has a resistance to tension in the undeployed configuration that is less than its resistance to tension in the deployed configuration. In the deployed configuration, the prosthesis is capable of twisting and bending. In one embodiment, the prosthesis has a meshwork of filaments woven, knitted or braided into a slender cylinder. In this embodiment, the prosthesis attains the deployed configuration by stretching the prosthesis from its undeployed configuration. The prosthesis may be used, for example, to replace an anterior or posterior cruciate ligament or to treat acromioclavicular joint separation, a rotator cuff tear, lateral collateral ligament tears, medial collateral ligament tears, or medial patello-femoral ligament tears. The invention also provides a method for replacing a tendon or ligament using the prosthesis of the invention.

### FIELD OF THE INVENTION

This invention relates to medical devices, and more specifically to such devices for compensating for a damaged tendon or ligament.

### BACKGROUND OF THE INVENTION

Tendons and ligaments have similar anatomical structures, but serve different biological functions. Both serve as load-bearing structures, but tendons attach muscle to bone, while ligaments attach bone to bone.

Occasionally, usually due to excessive stress, unnatural movement or injury, a ligament or tendon might tear, either partially or fully. One of the most common ligaments to tear is the anterior cruciate ligament (ACL) which joins the tibia to the femur. One generally accepted method of treatment for such a condition is replacing the torn ligament or tendon with an autograft. This involves harvesting another ligament or tendon from elsewhere in the body and transplanting it at the site of the torn ligament or tendon. Although this process is often successful, it can result in some loss of mobility at the donor location, as well as various other complications such as pain and local morbidity at the donor site. Another accepted method is using an allograft, which has its own drawbacks, such as risk of infectious diseases and high costs.

There have also been numerous attempts to develop a prosthetic replacement for damaged ligaments and tendons, for example, as disclosed in U.S. Pat. Nos. 4,642,119; 6,599,319; 4,792,336; 6,287,340 ;5,595,621 ;5,575,819 ;7,101,398; 4,755,183; 4,932,972; 5,004,474 and 5,197,983. The devices disclosed in there publications are based on polymeric materials or carbon fibers which undergo degradation inside the human body, due mainly to processes of aging, fatigue and water absorption from body fluids.

Another example of a prosthetic graft for treatment of torn tendons or ligaments is disclosed in U.S. Pat. No. 4,983,184. The device disclosed in that patent involves a graft for reinforcement of a torn tissue to allow healing.

One of the traits of a ligament or a tendon is its anisotropic behavior. It is relatively resistant to tension forces, but easily flexes under torsional and bending forces. Furthermore, during tension, a ligament or tendon exhibits large deformation before failure. Due to the un-crimping of collagen fibers and the elasticity of elastin, the initial portion of a ligament or tendon stress-strain curve has a high deformation/low force characteristic known as the toe region, which is non-linear. A linear region is typically identified after the toe region and is used for the determination of the elastic modulus. The elastic modulus varies for different ligaments or tendons in the body, and changes according to age and gender, so, for instance, the stiffness of an ACL in a young male may be 200 N/mm, and beyond the elastic region, a failure region is evident. Failure force also varies in different ligaments in the body, and in different people. For instance, the tensile yield force for of an ACL in a young male may be 450N.

### SUMMARY OF THE INVENTION

The present invention provides a tendon or ligament prosthesis. In accordance with the invention, the prosthesis of the invention has an undeployed configuration and a deployed configuration. The prosthesis in the deployed configuration can withstand a higher tension force than when in the undeployed configuration. Furthermore, in the deployed configuration the prosthesis is capable of bending and twisting.

The prosthesis of the invention preferably has mechanical properties that tend to mimic those of a tendon or ligament: a tensile stress-strain curve having a toe-region followed by a linear region, with high tensile stiffness, at least an order of magnitude greater than bending or torsional stiffness.

In one preferred embodiment, the prosthesis comprises a plurality of filaments arranged in a mesh. For example, the filaments may have a helical shape with the filaments woven into a slender cylinder. In this embodiment, the filaments are arranged in a loosely packed mesh structure in the undeployed configuration. This allows the prosthesis in the undeployed configuration to be stretched into its deployed configuration, up to a desired tension. As the prosthesis is stretched, the diameter of the prosthesis decreases and the packing of the filaments becomes denser. Thus, as the prosthesis is stretched, the filaments un-coil, similar to un-crimping of collagen fibers in a tendon or ligament, so that the resistance of the prosthesis to further stretching increases, mimicking the toe region of a natural tendon or ligament. Therefore, the prosthesis in the deployed configuration has higher stiffness to tension than in the undeployed configuration. Once the filaments are uncoiled, the prosthesis has an elastic modulus dependant on the material properties (Young's modulus) and the overall thickness of the filaments. However, due to the low moment of inertia of the thin filaments, in the deployed configuration the prosthesis is capable of bending and twisting. Since the tensile stiffness of the prosthesis is strongly dependant on the number of filaments, a desired stiffness value corresponding to that of the target ligament or tendon may be achieved by selecting an appropriate number of filaments in the prosthesis.

The prosthesis may be provided with an anchoring device at one or both ends for anchoring the ends to a body tissue, such as a bone tissue, or cartilage. The prosthesis may also be provided with a protective sleeve to reduce friction between the prosthesis and body structures after deployment of the prosthesis.

The prosthesis of the invention can be used for replacement of almost any tendon or ligament in a human or animal body. Uses of the prosthesis of the invention in human surgery include anterior cruciate ligament (ACL) reconstruction, posterior cruciate ligament (PCL) reconstruction, rotator cuff repair, acromioclavicular joint separation surgery, lateral collateral ligament (LCL) repair, medial collateral ligament (MCL) repair, medial patello-femoral surgery or any other damaged ligament or tendon. The device of the invention may be used as an anchor to connect parts of a damaged or torn tendon or ligament , or as a graft to replace a ligament or tendon. In veterinary surgery, uses of the prosthesis of the invention may include cranial cruciate ligament (CCL) to reconstruction in dogs or horses.

Thus, in its first aspect, the invention provides a tendon or ligament prosthesis having an undeployed configuration and a deployed configuration, the prosthesis having a resistance to tension in the undeployed configuration that is less than a resistance to tension of the prosthesis in the deployed configuration, and the prosthesis being capable of twisting and bending in the deployed configuration.

The invention also provides use of a prosthesis of the invention to treat acromioclavicular joint separation, a rotator cuff tear, lateral collateral ligament tears, medial collateral ligament tears or medial patello-femoral ligament tears.

The invention further provides a method for replacing a tendon or ligament having a first attachment site and a second attachment site comprising (a) attaching a first end of a prosthesis of the invention in the undeployed configuration to the first attachment site; (b) bringing the prosthesis to the deployed configuration; and (c) attaching a second end of the prosthesis in the deployed configuration to the second attachment site.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1a shows a tendon or ligament prosthesis in accordance with one embodiment of the invention in its undeployed configuration, and FIG. 1b shows the prosthesis of Fig. la in its deployed configuration;

FIG. 2 shows a tendon or ligament prosthesis in accordance with another embodiment of the invention;

FIGS. 3a to 3e show use of a tendon or ligament prosthesis in the replacement of an anterior cruciate ligament (ACL);

FIG. 4 shows use of a tendon or ligament prosthesis in rotator cuff repair; and

FIG. 5 shows use of an tendon or ligament prosthesis of the invention in acromioclavicular joint (ACJ) separation surgery; and

FIG. 6 shows use of a tendon or ligament prosthesis in the replacement of an anterior cruciate ligament (ACL).

### DETAILED DESCRIPTION OF EMBODIMENTS

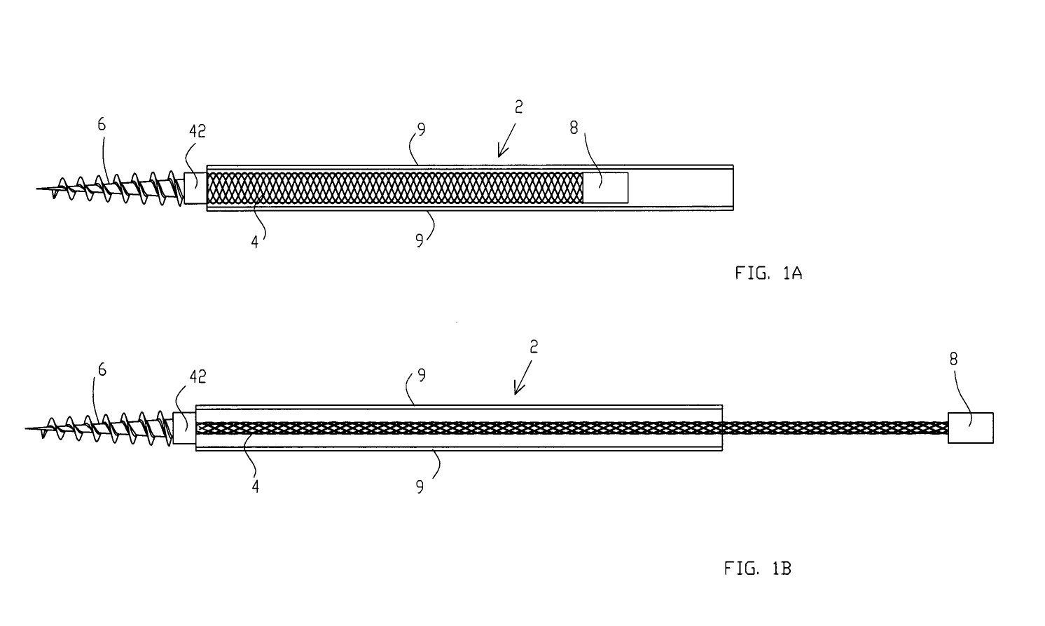

FIG. 1 shows a tendon or ligament prosthesis 2 in accordance with one embodiment of the invention. The prosthesis 2 has an undeployed configuration shown in FIG. 1a, and a deployed configuration shown in FIG. 1b. The prosthesis 2 comprises a plurality of filaments 4 arranged in an elongated mesh. In FIG. 1, each filament has a helical shape, and the filaments have been woven into a slender cylinder. The ends of the prosthesis may comprise an anchoring device for anchoring each end to a body tissue, such as a bone tissue, or cartilage. The anchoring device may be an interference screw 6 or a bone anchor 8. The prosthesis 2 may be provided with a protective sleeve 9 to reduce friction between the prosthesis and body structures after deployment of the prosthesis. The sleeve 9 may be made from a biodegradable material, such as poly glycolic acid, poly lactic acid, poly caprolactone, dioxanone, chondroitin sulphate, hyaluronic acid, or a synthetic polymer based on hyaluronic acid such as HYAFF®.

In its undeployed configuration (FIG. 1a) the filaments 4 are arranged in a loosely packed mesh structure. This allows the prosthesis in the undeployed configuration to be stretched to bring the prosthesis into its deployed configuration (FIG. 1b). As the prosthesis is stretched, the diameter of the prosthesis decreases and the packing of the filaments becomes denser. In the case of helically shaped filaments, the pitch of the helix increases during stretching. Thus, as the prosthesis is stretched, the resistance of the prosthesis to further stretching increases. Therefore, the prosthesis in the deployed configuration has a higher tensile stiffness than in the undeployed configuration. However, due to the low moment of inertia of the thin filaments, in the deployed configuration the prosthesis 2 is capable of bending and twisting about its longitudinal axis.

The filaments in the prosthesis are preferably made from a biocompatible metallic material, and most preferably from a metallic alloy such as stainless steel, an alloy of cobalt, an alloy of titanium, or Nitinol®. The stainless steel may be, for example, ASTM 138 (316L or 316 LVM.) or ASTM 2229 (Nickel free Stainless Steel). The cobalt alloy may be, for example, ASTM F75, ASTM F799, ASTM F790, ASTM F562 or ASTM F1058. The titanium alloy may be, for example, ASTM F67 (unalloyed Titanium) ASTM F136 (Ti-6Al-4Va) ASTM F1295 (Ti-6Al-7Nb) or ASTM F2066 (Ti-15Mo).

At least part of the filaments in the prosthesis may be made from a bioabsorbable metallic material such as magnesium alloys. In this form, the prosthesis is used not as a permanent implant, but rather as a temporary augmentation device, to allow for repair rather than replacement of a torn ligament or tendon.

In a most preferred embodiment, the prosthesis is made from filaments exhibiting super elastic properties at body temperature or under stress. Filaments made from Nitinol™ are particularly preferred since Nitinol™ allows higher strain rates than most other biocompatible alloys.

When provided with one or more anchoring devices, the anchoring devices may be made from a different metal or alloy than the filaments, but having similar galvanic properties (both materials close to each other on the galvanic scale) in order to prevent galvanic corrosion. Alternatively, galvanic corrosion may be prevented by making the anchoring devices from a non-metallic biocompatible material such as zirconia.

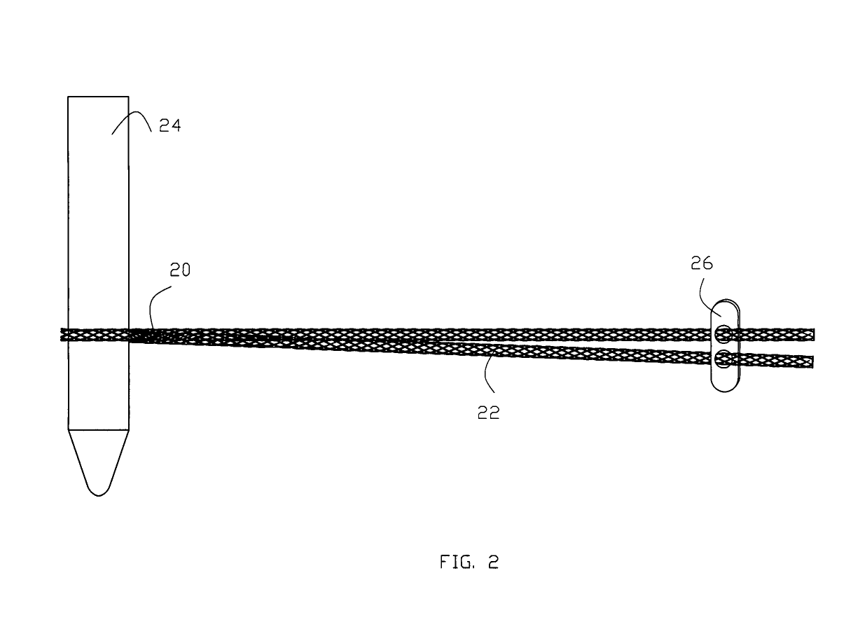

FIG. 2 shows a tendon or ligament prosthesis 20 in accordance with another embodiment of the invention. As with the prosthesis 2 (FIG. 1), the prosthesis 20 comprises a plurality of filaments 22 are arranged in a loosely packed mesh structure to form a slender cylinder. This arrangement of the filaments 22 allows the prosthesis to be stretched from its undeployed configuration to its deployed configuration. The prosthesis 20 is provided with a cross pin 24 for anchoring an end of the prosthesis to a body tissue, such as a bone tissue, or cartilage. Another end of the prosthesis 20 can be anchored to a body tissue by means of a suture button 26. The prosthesis 20 may be provided with a protective sleeve (not shown).

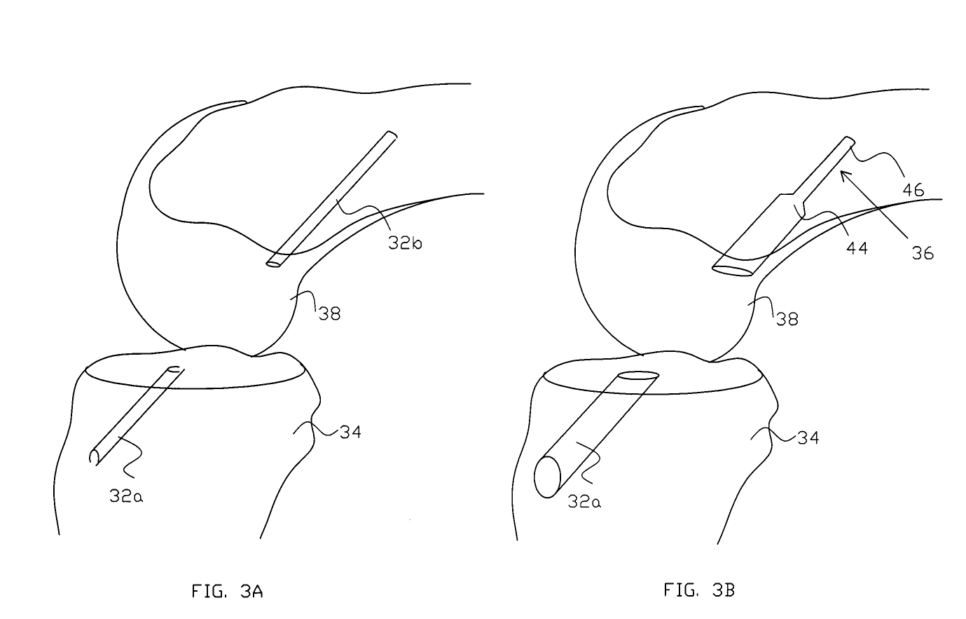

FIG. 3 shows use of a prosthesis of the invention in the replacement of an anterior cruciate ligament (ACL). As shown in FIG. 3a, a pilot tunnel 32a is drilled through the tibia 34, and a blind hole 32b is drilled through the femur 38, where the tunnel 32a and the hole 32b are collinear. Then, as shown in FIG. 3b, the tunnel 32a is redrilled to widen it, and the hole 32b is widened at its opened end to form a stepped hole 36 having a broad portion 44 proximal to the knee joint and a narrow portion 46 distal to the knee joint. With the prosthesis 2 in its undeployed configuration, the interference screw 6 is pushed through the tunnel 32a and into the broad portion 44 of the hole 36. The bone anchor 6 is then screwed into the narrow portion 46 of the hole 36 (FIG. 3c). In this way, the head 42 of the bone anchor screw 6 is countersunk in the broad portion 44 of the stepped tunnel 36. The threaded tip 48 is engaged in the narrow portion 46 of the stepped tunnel 36. After attachment of the bone screw 6 in the stepped tunnel 36, the prosthesis 30 is stretched to its deployed configuration (FIG. 3d). After stretching to achieve a desired tensile strength, the prosthesis may be cut in situ to the appropriate length, and tied into a knot 68 at its free end and restrained on the surface of the tibia 34 by means of a suture button 69 (FIG. 3e).

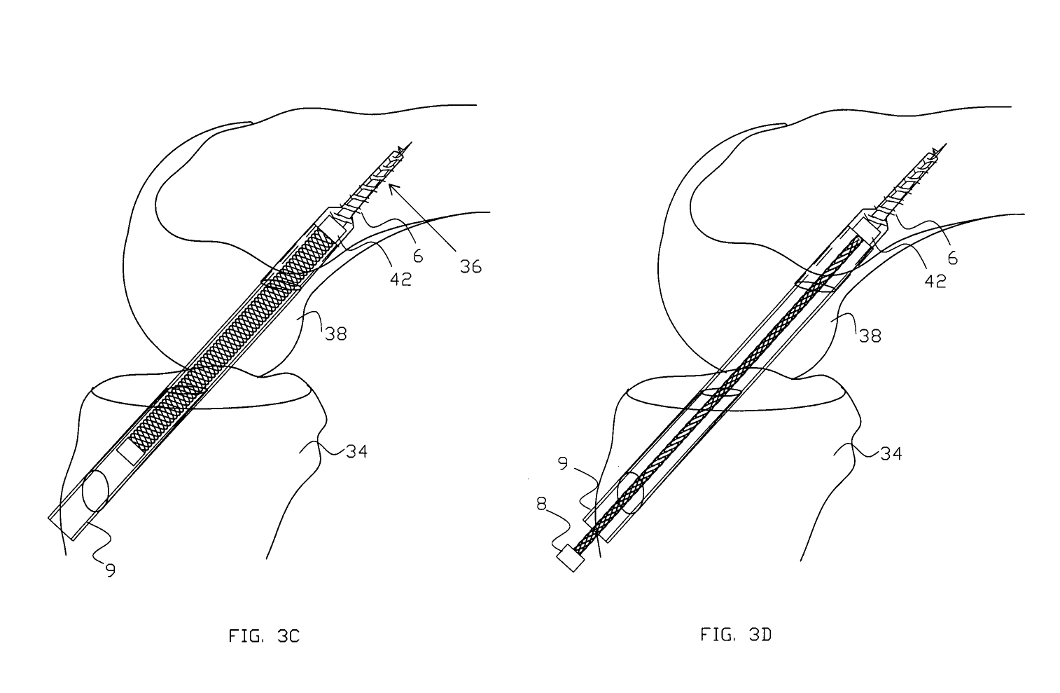

FIG. 4 shows deployment of a prosthesis 50 of the invention to repair a torn rotator cuff. The prosthesis 50 has at one end a bone anchor 6 that is screwed into the humerus 51 with the head 42 of the screw 6 countersunk below the surface of the humerus, as explained above in reference to FIG. 3. After screwing the anchor 6 into the humerus 51 , the prosthesis is stretched into its deployed configuration and sutured to the rotator cuff 52.

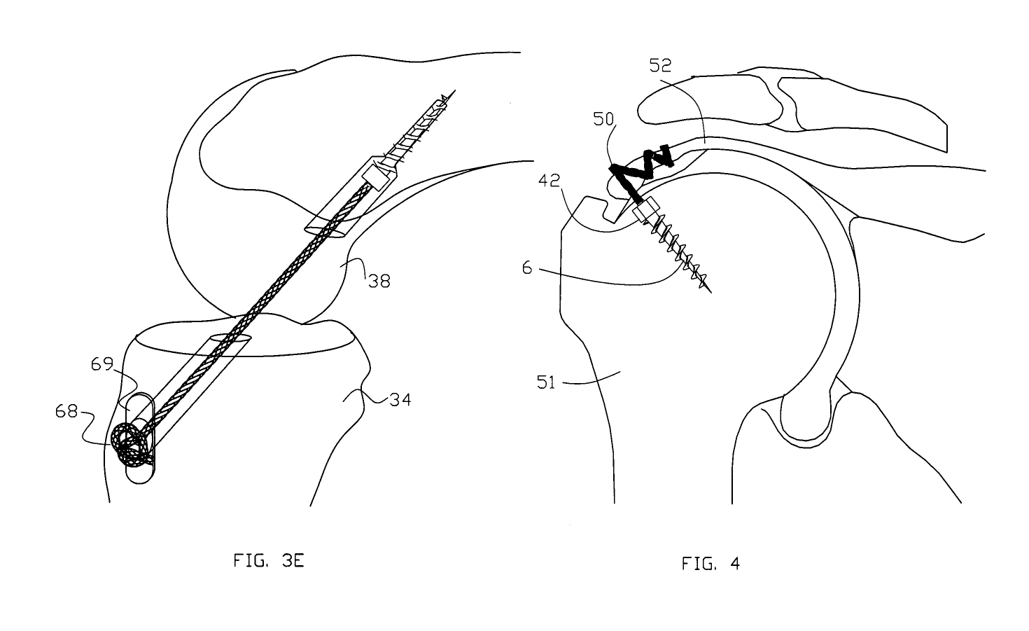

FIG. 5 shows use of a prosthesis 62 of the invention in replacement of an entire ligament or tendon in acromioclavicular joint (ACJ) separation surgery. A tunnel 64 is drilled through the clavicle 63 , and a stepped hole 65 is drilled into the scapula 61. A bone anchor 6 is inserted through the tunnel 64 and into the stepped hole 65 and the anchor 6 is screwed in the stepped tunnel and is countersunk below the surface of the scapula 61. The prosthesis 62 is then stretched into its deployed configuration and a suture button 68 is used to secure the other end of the prosthesis 62 at the surface of the clavicle 63.

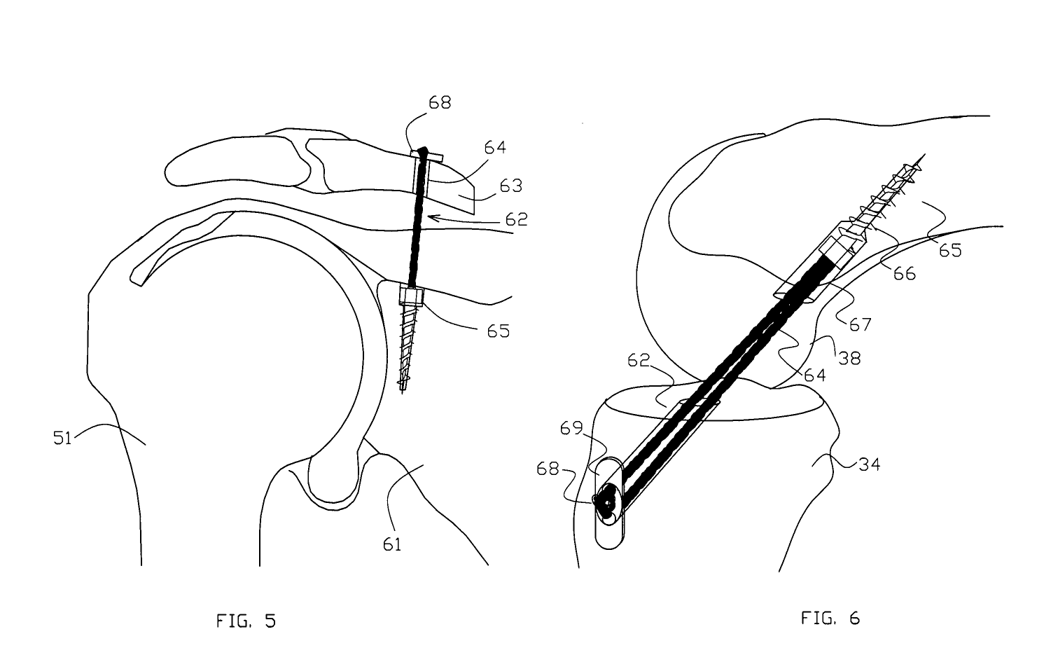

FIG. 6 shows use of a prosthesis 60 for replacement of a ligament or tendon such as an anterior cruciate ligament (ACL). The prosthesis has a first woven bundle 62 of fibers and a second woven bundle of fibers 64 parallel to the first bundle. As with the embodiment of FIG. 3, a tunnel 62 is drilled in the tibia 34 and a blind stepped hole 67 is drilled through the femur 38. With the prosthesis in its undeployed configuration, a bone anchor 66 at one end of the prosthesis 60 is pushed through the tunnel 65 and is then screwed into the narrow portion of the hole 67 with the head of the bone anchor 66 countersunk below the surface of the femur 38. After attachment of the interference screw 66 in the stepped hole 67, the prosthesis is stretched to its deployed configuration to achieve a desired tensile strength. The prosthesis may be cut in situ to the appropriate length, and the free ends tied together into a knot 68 and immobilized on the surface of the tibia 34 by means of a suture button 69.

1. A tendon or ligament prosthesis having an undeployed configuration and a deployed configuration, the prosthesis having a resistance to tension in the undeployed configuration that is less than a resistance to tension of the prosthesis in the deployed configuration, and the prosthesis being capable of twisting and bending in the deployed configuration.

2. The prosthesis according to claim 1 comprising a meshwork of filament.

3. The prosthesis according to claim 2 wherein the filaments are braided, woven or knitted into the meshwork.

4. The prosthesis according to claim 2 wherein the meshwork comprises helically shaped filaments.

5. The prosthesis according to claim 1, wherein at least one of the filaments comprises a metal or an alloy.

6. The prosthesis according to claim 5 wherein the alloy is biodegradable.

7. The prosthesis according to claim 5 wherein the alloy is stainless steel.

8. The prosthesis according to claim 5 wherein the alloy comprises cobalt.

9. The prosthesis according to claim 5 wherein the alloy comprises titanium.

10. The prosthesis according to claim 5 wherein the alloy is Nitinol™.

11. The prosthesis according to claim 1, wherein the prosthesis is configured to attain the deployed configuration by stretching.

12. The prosthesis according to claim 1, further comprising an attachment element at at least one end of the prosthesis.

13. The prosthesis according to claim 12 wherein the attachment element is a bone anchor, an interference screw, a cross pin or a suture button.

14. The prosthesis according to claim 1, further comprising a polymeric sleeve.

15. The prosthesis according to claim 14 wherein said polymeric sleeve is biodegradable.

16. The prosthesis according to claim 14 wherein said polymeric sleeve comprises any one or more of poly glycolic acid, poly lactic acid, poly caprolactone, dioxanone, chondroitin sulphate, hyaluronic acid or a synthetic polymer based on hyaluronic acid.

17-22. (canceled)

23. A method for replacing a tendon or ligament having a first attachment site and a second attachment site comprising (a) attaching a first end of a prosthesis according to claim 1 in the undeployed configuration to the first attachment site; (b) bringing the prosthesis to the deployed configuration; and (c) attaching a second end of the prosthesis in the deployed configuration to the second attachment site.

24. The method according to claim 23, wherein the tendon or ligament is a rotator cuff, an anterior or posterior cruciate ligament, a lateral collateral ligament, a medial collateral ligament or a medial patello-femoral ligament.

25. The method according to claim 23, which is conducted in acromioclavicular joint surgery.
